(19) United States
(12) Patent Application Publication
(10) Pub. No.: US 2026/0148248 A1
(43) Pub. Date: May 28, 2026

(54) Transaction Support Device, Transaction Support Program, and Transaction Support Method

(71) Applicant: 2SLAB INC., Chiba (JP)

(72) Inventor: Yohei SAKAMOTO, Chiba (JP)

(21) Appl. No.: 19/485,671

(22) PCT Filed: Jun. 22, 2023
(86) PCT No.: PCT/JP2023/023140

§ 371 Date: Nov. 18, 2025

(51) Int. Cl. G06Q 30/018; G06Q 40/04; G16B 30/00

(52) U.S. Cl. CPC G06Q 30/018; G06Q 40/055; G16B 30/00; G06Q 2220/10

(57) ABSTRACT

To support transactions while guaranteeing that an organism indeed possesses the genetic information with which it is associated, a transaction support device for supporting transactions of an organism, the device comprises a genetic information storage unit configured to store genetic information of the organism, and an NFT issuance unit configured to generate at least one of a pedigree certificate and digital art based on the genetic information, and to record, in a blockchain network, a user ID identifying a holder of the organism in association with a non-fungible token (NFT) associated with the at least one of the pedigree certificate and the digital art.

### TECHNICAL FIELD

The present invention relates to a transaction support device, a transaction support program, and a transaction support method.

### BACKGROUND ART

Methods have been proposed for selling a living organism whose genetic information has been identified.

For example, Patent Literature 1 discloses that genetic trait identifiers such as nucleic acid sequences are associated with animals, embryos, gametes, and the like.

### PRIOR-ART DOCUMENTS

### Patent Literature

[Patent Literature 1] JP Patent Application Publication No. 2009-518712

### SUMMARY OF THE INVENTION

### Problem to be Solved by the Invention

However, the technique disclosed in Patent Literature 1 has no mechanism for guaranteeing that the genetic trait identifier linked to an animal or the like is correct; therefore, it can occur that an animal or the like purchased by a user does not possess the genetic trait in question.

Accordingly, an object of the present invention is to support transactions that include a mechanism for guaranteeing that a living organism indeed possesses the genetic information with which it is associated.

### Means for Solving the Problems

According to one aspect of the present invention, a transaction support device for products includes: a genetic information storage unit that stores genetic information of the organism; and an NFT issuance unit that generates a pedigree certificate and digital art based on the genetic information, and records, in a blockchain network, a user ID identifying a holder of the organism in association with a non-fungible token (NFT) associated with the pedigree certificate and the digital art.

### Effects of the Invention

According to the present invention, transactions can be supported while guaranteeing that the living organism indeed possesses the genetic information with which it is associated.

### BRIEF DESCRIPTION OF THE DRAWINGS

FIG. 1 is a block diagram illustrating a transaction support system according to the present embodiment.

FIG. 2 is a block diagram illustrating a hardware configuration of server device 1 according to the present embodiment.

FIG. 3 is a block diagram illustrating a functional configuration of server device 1 according to the present embodiment.

FIG. 4 is a diagram illustrating an example of user information stored in a user information storage unit 131 according to the present embodiment.

FIG. 5 is a diagram illustrating an example of organism information stored in an organism information storage unit 132 according to the present embodiment.

FIG. 6 is a diagram illustrating an example of genetic information stored in a genetic information storage unit 133 according to the present embodiment.

FIG. 7 is a diagram illustrating an example of pedigree certificate information stored in a pedigree certificate storage unit 134 according to the present embodiment.

FIG. 8 is a diagram illustrating an example of a pedigree certificate generated by a pedigree certificate generation unit 114 according to the present embodiment.

FIG. 9 is a diagram illustrating an example of art generated by an art generation unit 115 according to the present embodiment.

FIG. 10 is a diagram illustrating an operational example of the transaction support system according to the present embodiment.

### EMBODIMENTS

Hereinafter, embodiments of the present invention will be described with reference to the drawings. It is to be understood that the embodiments described below are not intended to unduly limit the scope of the invention as set forth in the claims. Further, not all of the components shown in the embodiments are necessarily essential components of the present invention.

### SUMMARY OF INVENTION

### Summary of the Invention

The content of the embodiments of the present invention is described below. The present invention, for example, comprises the following configuration.
[Item 1] A transaction support device for supporting transactions of organisms, the device comprising:
a genetic information storage unit configured to store genetic information of the organism; and
an NFT issuance unit configured to generate at least one of a pedigree certificate and digital art based on the genetic information, and to record, in a blockchain network, a user ID identifying a holder of the organism in association with a non-fungible token (NFT) associated with at least one of the pedigree certificate and the digital art.
[Item 2] The transaction support device according to Item 1, wherein the NFT issuance unit is configured to generate the digital art based on information of a nucleotide sequence included in the genetic information.
[Item 3] The transaction support device according to Item 1 or 2, comprising a trait simulation unit configured to predict a trait of offspring when a first organism and a second organism are crossed, based on trait information of the first organism and the second organism and on the genetic information.
[Item 4] The transaction support device according to Item 3, comprising a distribution unit configured to, when revenue is obtained by allowing a viewing user who wishes to view the pedigree certificate to view the pedigree certificate, distribute a portion of the revenue to an inspection user who is the owner of the pedigree certificate.
[Item 5] A transaction support program for supporting transactions of organisms, the program causing a processor to execute:
a genetic information storing step of storing genetic information of the organism; and
an NFT issuing step of generating at least one of a pedigree certificate and digital art based on the genetic information, and recording, in a blockchain network, a user ID identifying a holder of the organism in association with an NFT associated with at least one of the pedigree certificate and the digital art.
[Item 6] A transaction support method for supporting transactions of organisms, the method comprising, by a processor:
storing genetic information of the organism; and
generating at least one of a pedigree certificate and digital art based on the genetic information, and recording, in a blockchain network, a user ID identifying a holder of the organism in association with an NFT associated with at least one of the pedigree certificate and the digital art.

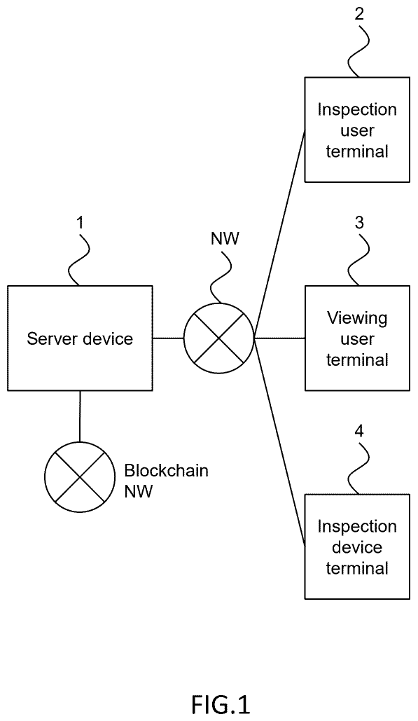

FIG. 1 is a diagram illustrating an example overall configuration of a transaction support system according to one embodiment of the present invention. The system provided in this embodiment includes server device 1. Server device 1 is communicably connected, via a communication network NW, to an inspection user terminal 2, a viewing user terminal 3, and an inspection device terminal 4. The communication network NW is, for example, the Internet, and is constructed using a public switched telephone network, a mobile telephone network, a wireless communication path, Ethernet (registered trademark), and the like.

Server device 1 is also communicably connected to a blockchain network (Blockchain NW in FIG. 1). The blockchain network is composed of a plurality of nodes (computers) and is capable of managing ledger data in a distributed manner. The distributed ledger is managed so as to be tamper-resistant by a so-called blockchain mechanism. The detailed description of the mechanism for distributed-ledger management by blockchain is omitted here, assuming adoption of a generally known scheme. The blockchain network can be constructed using, for example, Ethereum.

Tokens (Ethereum tokens) that can be issued on a blockchain network such as Ethereum include fungible tokens and non-fungible tokens (NFTs). An NFT is, for example, a token issued under the “ERC-721” standard on Ethereum, which is a blockchain-network platform (also referred to as an ERC-721 NFT); it is a unit of data recorded on the blockchain network and, based on a unique identifier (NFT ID), can be associated with an edition (ownership) of a digital item, thereby having a non-fungible character. An NFT can have a unique value distinct from other NFTs. In the present embodiment, by way of example, the NFT is assumed to be an ERC-721 NFT. Like fungible tokens, NFTs are tradable on the blockchain network. Transaction histories of NFTs are recorded on the blockchain network. In the distributed ledger of the blockchain network, the owner of an NFT and its ownership history are also recorded.

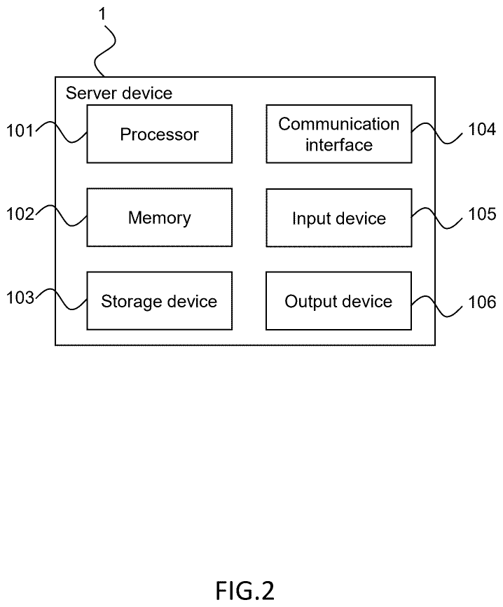

FIG. 2 is a diagram illustrating an example hardware configuration of server device 1. The configuration shown is merely an example, and other configurations may be used. Server device 1 includes a CPU 101, a memory 102, a storage device 103, a communication interface 104, an input device 105, and an output device 106. The storage device 103 is, for example, a hard disk drive, a solid-state drive, or flash memory that stores various data and programs. The communication interface 104 is an interface for connection to communication network 2 and may be, for example, an adapter for connection to Ethernet (registered trademark), a modem for connection to a public switched telephone network, a wireless communication device for wireless communications, or a USB (Universal Serial Bus) connector or an RS-232C connector for serial communications. The input device 105 is, for example, a keyboard, a mouse, a touch panel, buttons, or a microphone for entering data. The output device 106 is, for example, a display, a printer, or a speaker for outputting data. The functional units of server device 1 described below are implemented when the CPU 101 reads a program stored in the storage device 103 into the memory 102 and executes it, and the storage units of server device 1 are implemented as portions of the storage area provided by the memory 102 and the storage device 103.

Server device 1 can receive genetic information of an organism from the inspection device terminal 4 and record, in the blockchain network, a non-fungible token (hereinafter, “NFT”) associated with an identifier of the organism or the like. The NFT is, for example, a token issued under the ERC-721 standard on Ethereum, which is a blockchain-network platform; it is a unit of data recorded on the blockchain network and, based on an ID, can be associated with an edition (ownership) of a digital item, thereby having a non-fungible nature. The NFT is recorded on the blockchain together with a smart contract and, being traceable, can certify transaction information including details and histories of, for example, the holder of the organism and the inspector of the genetic information. In addition, by using a smart contract, contracts regarding transactions between a platform operator and a user, or among multiple users, can be automatically generated, approved, and executed without a third party.

### Inspection User Terminal 2

The inspection user terminal 2 is a computer handled by a user who wishes to have an organism's genetic information inspected (hereinafter, “inspection user”), for example, the organism's owner. Examples include a smartphone, a tablet computer, or a personal computer. The inspection user can access server device 1, for example, via an application or a web browser running on the inspection user terminal 2.

### Viewing User Terminal 3

The viewing user terminal 3 is a computer handled by a user who wishes to view genetic information or a pedigree certificate of an organism (hereinafter, “viewing user”). Examples include a smartphone, a tablet computer, or a personal computer. The viewing user can access server device 1, for example, via an application or a web browser running on the viewing user terminal 3.

Note that the inspection user may also serve as the viewing user.

### Inspection Device Terminal 4

The inspection device terminal 4 is a computer that controls an inspection device which performs inspection of an organism's genetic information. Examples include a smartphone, a tablet computer, or a personal computer. A user of the inspection device terminal 4 can access server device 1, for example, via an application or a web browser running on the inspection device terminal 4.

The inspection device in this embodiment is a device that inspects genetic information of an organism, and may be, for example, a DNA sequencer, a next-generation sequencer, or a DNA microarray analyzer, but is not limited thereto.

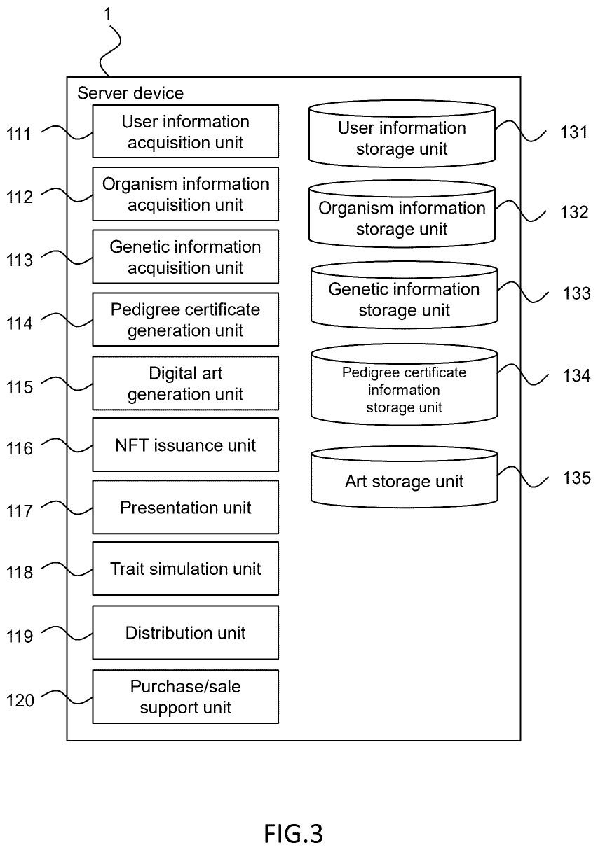

FIG. 3 is a functional block diagram of server device 1 in FIG. 1. Server device 1 includes the following processing units: a user information acquisition unit 111, an organism information acquisition unit 112, a genetic information acquisition unit 113, a pedigree certificate generation unit 114, a digital art generation unit 115, an NFT issuance unit 116, a presentation unit 116, a trait simulation unit 118, a distribution unit 119, and a purchase/sale support unit 120; and the following storage units: a user information storage unit 131, an organism information storage unit 132, a genetic information storage unit 133, a pedigree certificate information storage unit 134, and an art storage unit 135.

Information stored in each of the storage units—the user information storage unit 131, the organism information storage unit 132, the genetic information storage unit 133, the pedigree certificate information storage unit 134, and the art storage unit 135—is described below.

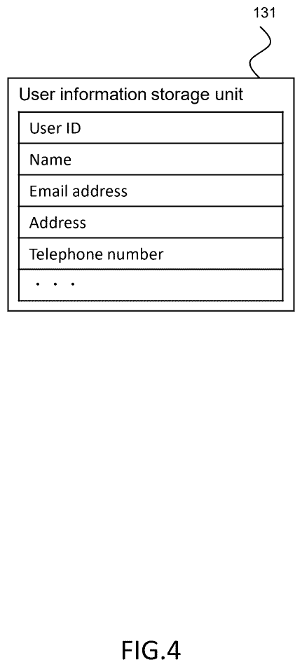

The user information storage unit 131 stores information about inspection users and viewing users acquired by the user information acquisition unit 111. As shown in FIG. 4, the user information includes, by way of example and without limitation, a name, email address, address, telephone number, bank account, wallet address, and credit card information, associated with a user ID.

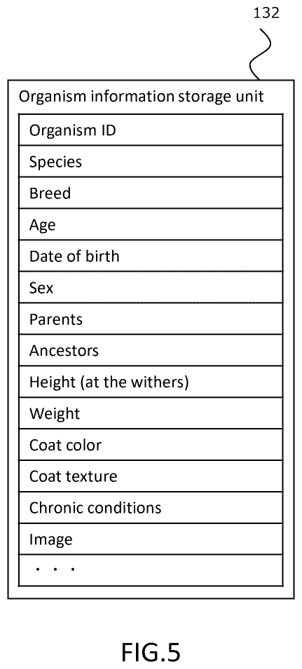

The organism information storage unit 132 stores information about organisms possessed by an inspection user (hereinafter, also referred to as an individual) acquired by the organism information acquisition unit 112. As shown in FIG. 5, the organism information includes, by way of example and without limitation, species, breed, name, date of birth, age, sex, parents, ancestors, height (at the withers), weight, coat color, coat texture, chronic conditions, and images of the organism, associated with an organism ID.

In this embodiment, the organism may be, for example, a pet, livestock, or farmed fish, but is not limited thereto. The inspection user may be, for example, a pet owner, livestock operator, or aquaculture operator, but is not limited thereto.

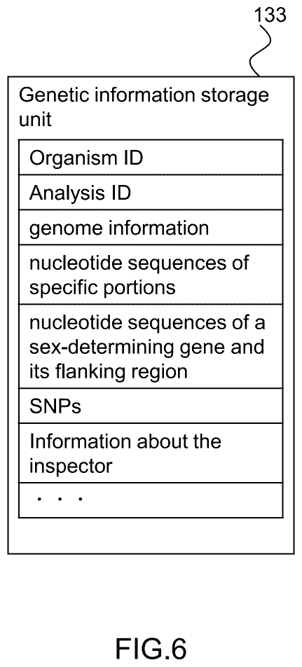

The genetic information storage unit 133 stores information relating to the genetic information of an organism acquired by the genetic information acquisition unit 113. As shown in FIG. 6, the genetic information includes, by way of example and without limitation, genome information, nucleotide sequences of specific portions, nucleotide sequences of a sex-determining gene and its flanking region, SNPs (single nucleotide polymorphisms), and information about the inspector, associated with an organism ID. The genetic information may be in formats such as a FASTA file, FASTQ file, sra file, abl file, or text file, without limitation.

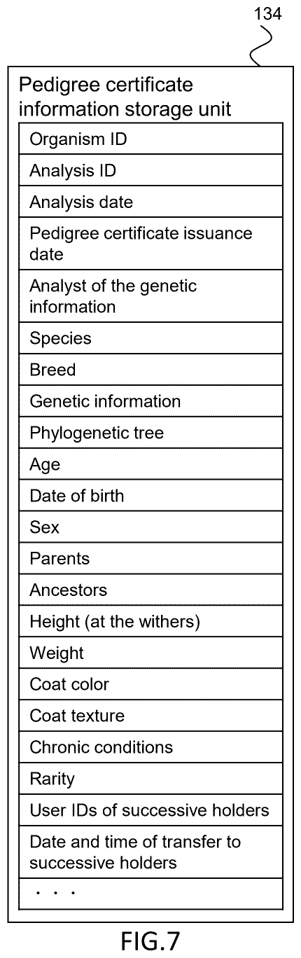

The pedigree certificate information storage unit 134 stores information of a pedigree certificate generated by the pedigree certificate generation unit 114. As exemplified in FIG. 7, the pedigree certificate information includes, by way of example and without limitation, species, breed, name, date of birth, age, sex, parents, ancestors, height (at the withers), weight, coat color, coat texture, chronic conditions, analysis ID, genetic information, a phylogenetic tree, information about the analyst of the genetic information, rarity, user IDs of successive holders, and the date and time of transfer to successive holders, associated with a user ID or organism ID.

The art storage unit 135 stores art generated by the art generation unit 115.

In this embodiment, art includes images, video, audio, and the like, and may include artworks, characters, and digital items (hereinafter collectively, “content”), without limitation. The ownership of the art can have a nature that allows sharing among multiple users. The ownership may be offered for sale as multiple editions (e.g., an edition size of 100), and each edition is associated with an NFT ID. Art purchased by a viewing user can be resold to other users by a method such as auction, and upon each acquisition of ownership by the viewing user, it is also possible to display the user's name on digital signage provided together with the art and on a web page related to the digital item.

The art in this embodiment may be provided to the inspection user who is the owner of the organism linked to the art, or to the viewing user who has purchased the organism, or to a third party who so desires. The viewing user may be allowed to select the purchase of the digital art when purchasing the organism.

It is also possible to provide all or part of the pedigree certificate information storage unit 134 and the art storage unit 135 on the blockchain network, thereby managing all or part of the pedigree certificate information and art information with a distributed ledger. For example, among the information stored in the pedigree certificate storage unit 134, the organism ID and genetic information may be stored on the blockchain network, whereas information such as species and breed may be stored on a storage medium of server device 1.

When a cavity is in its resonance condition, an exact integer number of wavelengths of light fits within the cavity, resulting in constructive laser light being stored within the cavity. When the cavity is in an anti-resonant condition, an exact half-integer number of wavelengths fits within the cavity resulting in destructive interference of the contained laser light, preventing laser energy from being stored within said cavity in an example.

The user information acquisition unit 111, by way of example, presents, via the network NW, an input form concerning user information to the inspection user terminal 2 or the viewing user terminal 3, and acquires the user information based on the user's input. The communications for such transmission and reception may be wired or wireless, and any communication protocol may be used as long as mutual communication can be carried out.

The organism information acquisition unit 112, by way of example, presents, via the network NW, an input form concerning organism information to the inspection user terminal 2, and acquires the organism information based on input from the inspection user. The communications for such transmission and reception may be wired or wireless, and any communication protocol may be used as long as mutual communication can be carried out.

The organism information acquisition unit 112, by way of example, may analyze an acquired image of the organism using known image analysis techniques and determine the species, breed, and traits. In addition, the organism information acquisition unit 112 may cross-check the information determined from the image with organism information received as input from the user, and, for any items determined to be erroneous, may request confirmation from the inspection user.

It is to be noted that the organism information acquisition unit 112 may, by way of example, also acquire a request for inspection of genetic information of the organism. When the organism information acquisition unit 112 acquires such a request, a business operator that conducts business using server device 1 may ship to the inspection user who made the request a kit for obtaining a sample for inspection (for example, a portion of a tail fin or another portion of the organism); the inspection user then returns the obtained sample to the business operator to hand over the sample, and the business operator, or a third party contracted by the business operator, performs the inspection of the genetic information. Since the method for inspecting genetic information may use existing methods, description thereof is omitted.

The genetic information acquisition unit 113, by way of example, acquires genetic information from the inspection device terminal 4 via the network NW. The communications for such transmission and reception may be wired or wireless, and any communication protocol may be used as long as mutual communication can be carried out. In addition, the genetic information acquisition unit 113 may acquire the genetic information by having a business operator that conducts business using server device 1 transfer genetic information stored in the inspection device terminal 4 to server device 1 using a storage medium or the like. Further, the genetic information acquisition unit 113 may acquire genetic information that the inspection user already possesses from the inspection user terminal 2.

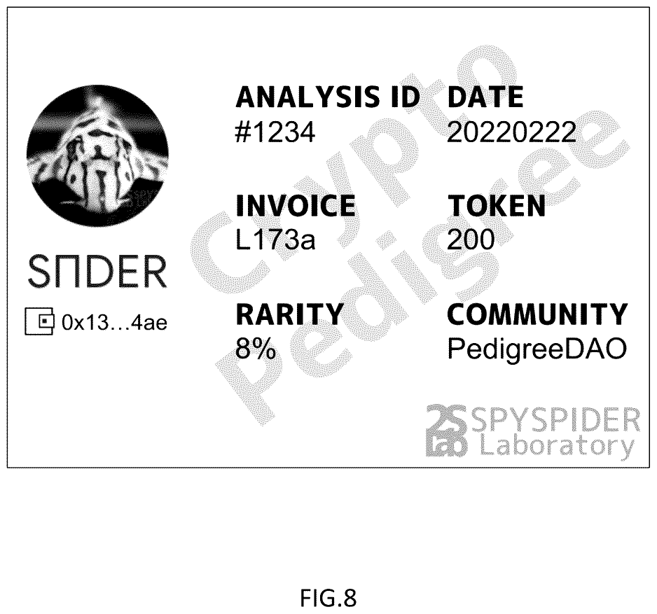

The pedigree certificate generation unit 114 generates a pedigree certificate corresponding to an organism. For example, the pedigree certificate generation unit 114 generates a pedigree certificate corresponding to the organism based on the organism information and the genetic information of a given organism. FIG. 8 shows an example of a pedigree certificate generated by the pedigree certificate generation unit 114.

The pedigree certificate generation unit 114 may generate figures produced based on genetic information, such as a phylogenetic tree or a plot of genotyping results, and may generate a pedigree certificate including information of such figures.

The pedigree certificate generation unit 114 may calculate a rarity of an organism and generate a pedigree certificate including information on the rarity. For example, the pedigree certificate generation unit 114 may calculate the rarity from a fixation rate based on organism information or genetic information of a given organism. In one example, a known probability that a phenotype such as coloration included in the organism information associated with an organism appears is stored as a fixation rate in server device 1, and the information of the fixation rate may be used as the rarity. When the organism has a plurality of phenotypes for which fixation rates are stored, the pedigree certificate generation unit 114 may calculate the rarity by multiplying the fixation rate associated with each phenotype. In addition, when the genetic information includes variants of SNPs known to be related to a particular phenotype, the probability that the SNP variants appear is stored in server device 1 as a fixation rate, and the information of the fixation rate may be used as the rarity. When the organism has a plurality of SNP variants for which fixation rates are stored, the pedigree certificate generation unit 114 may calculate the rarity by multiplying the fixation rate associated with each SNP variant. The pedigree certificate generation unit 114 may calculate the rarity based on a fixation rate A for a phenotype included in the organism information and a fixation rate B for SNP variants known to be related to a particular phenotype included in the genetic information. In this case, the pedigree certificate generation unit 114 may calculate the rarity by multiplying fixation rate A and fixation rate B, or may calculate the rarity by multiplying each of them after multiplying fixation rate A or fixation rate B by a predetermined coefficient.

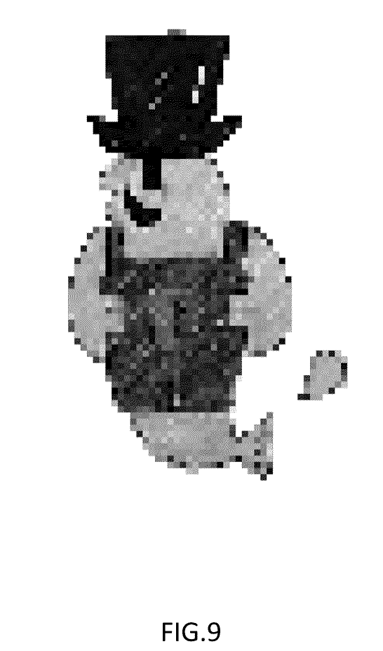

The digital art generation unit 115 generates digital art based on information of the pedigree certificate. For example, the digital art generation unit 115 generates art based on the organism information and the genetic information of the organism on which the pedigree certificate is based. FIG. 9 shows an example of art generated by the digital art generation unit 115. The digital art generation unit 115, for example, arranges the nucleotide bases A, T, C, and G in a frame shape corresponding to the species and breed stored in server device 1, based on species and breed information included in the organism information associated with the organism on which the pedigree certificate is based. Further, based on flanking sequences that include SNPs specific or characteristic to the organism and included in the genetic information associated with the organism, the digital art generation unit 115 may replace the colors of A, T, C, and G with four colors, or, based on the positions/number of the SNPs or on individual coloration included in the organism information, may change the eye color or the base tone, but is not limited to these methods.

The digital art generation unit 115 may, for example, generate art based on traits that may appear in offspring of the organism on which the pedigree certificate is based, using the organism information and genetic information of that organism.

When the pedigree certificate generation unit has generated a pedigree certificate, the NFT issuance unit 116 issues an NFT corresponding to the pedigree certificate (which certifies the owner of the pedigree certificate (the owner of the organism)). Information identifying the pedigree certificate and information identifying the owner (user ID), and the like, are set in the NFT to be issued. In addition, the NFT issuance unit 116 may issue an NFT corresponding to art generated by the digital art generation unit.

The NFT issuance unit 116 can send the issued NFT to the wallet of the inspection user who is the owner.

The presentation unit 117 presents to the inspection user the pedigree certificate generated by the pedigree certificate generation unit 114 and the digital art generated by the digital art generation unit 115.

The presentation unit 117 may grant the inspection user access rights to the pedigree certificate information storage unit 134 and the digital art storage unit 135, and present a URL or the like.

The presentation unit 117 presents to the inspection user the pedigree certificate generated by the pedigree certificate generation unit 114 and the digital art generated by the digital art generation unit 115.

The presentation unit 117 acquires from the viewing user a request to view a specified pedigree certificate or art. Together with the request to view, the presentation unit 117 may acquire a payment (viewing fee) from the viewing user in a specified currency or cryptocurrency, and the like. When the presentation unit 117 has acquired the request to view or the payment (viewing fee) from the viewing user, it may grant access rights to the pedigree certificate information storage unit 134 and the digital art storage unit 135, and present a URL or the like.

The trait simulation unit 118 performs a simulation of phenotypic traits in the case where the organisms underlying the pedigree certificates are crossed, based on information from multiple pedigree certificates. The trait simulation unit 118 acquires, from the inspection user or the viewing user, for example, a request for trait simulation that specifies two pedigree certificates. For example, based on the information of the specified pedigree certificates, the trait simulation unit 118 may perform a simulation of phenotypic traits in the case where the organisms underlying those pedigree certificates are crossed.

The trait simulation unit 118 may, for example, simulate, based on scientifically established facts, what traits such as coloration, body type, body length, height (at the withers), and physical characteristics would be in the case where the genetic information included in the specified pedigree certificates is combined. In addition, probabilities such as the probability that variants are introduced into the genetic information due to meiotic division of germ cells or recombination may be taken into account, and probability information may be added to the simulation results. The trait simulation unit 118 may also simulate next-generation traits by combining the genetic pedigree background within the species in the current generation (e.g., phylogenetic analysis and STRUCTURE analysis) with trait information.

The trait simulation unit 118 may, for example, use as the simulation result a trait in cases where: the specified pedigree certificates include information on a special individual (for example, an individual whose phenotype such as coloration, body type, body length, or height (at the withers) differs from that of a wild-type individual); the gene causing the phenotype has been identified; and scientific information such as the frequency has been clarified. For example, L236 (invoice name), a type of pleco (ornamental fish), has progressed in breeding improvement, so the fixation rate of variant-type traits (special individuals) in the next generation is high; whereas L333 (invoice name) is known to still retain many wild-type traits, so the fixation rate of variant-type traits (special individuals) in the next generation tends to be low. However, regardless of the breed, because it is possible to produce a stabilized strain having variant-type traits by pursuing breeding improvement by selective breeding, the trait simulation unit 118 may simulate (predict) next-generation traits by combining the genetic pedigree background within the species in the current generation (e.g., phylogenetic analysis and STRUCTURE analysis) with trait information.

When the trait simulation unit 118 accepts a specification of a pedigree certificate that includes information on a special individual (for example, an individual whose phenotype-such as coloration, body type, body length, or height (at the withers) differs from that of a wild-type individual), and the gene causing the phenotype has not been identified, the unit may, for example, take into account the breed of the parents, the phenotype (appearance, etc.), and the rate of variant individuals (fixation rate) in offspring, and return a simulation result such as “there is a high likelihood that S/A/B/C/D-grade next-generation offspring will appear.”

The trait simulation unit 118 may, for example, acquire a payment (simulation fee) from the user who made the request for trait simulation, in a specified currency or cryptocurrency, and the like.

When a payment is obtained for viewing a pedigree certificate or for trait simulation, the distribution unit 119 may distribute a portion of the payment to the inspection user who is the owner of the pedigree certificate that was viewed or that was the subject of the trait simulation.

The sales support unit 120 supports sales of organisms. The sales support unit 120 may be provided with a chat system or a bulletin board so that communication can be made between the inspection user and the viewing user. The sales support unit 120 may accept, from the inspection user terminal 2, conditions for selling an organism (price, delivery method, etc.) and present them to the viewing user terminal 3. The sales support unit 120 accepts a purchase application for an organism from the viewing user and presents it to the inspection user. When accepting a purchase application from the viewing user, the sales support unit 120 may request a payment, and pay to the inspection user an amount obtained by deducting a fee (commission) from the payment.

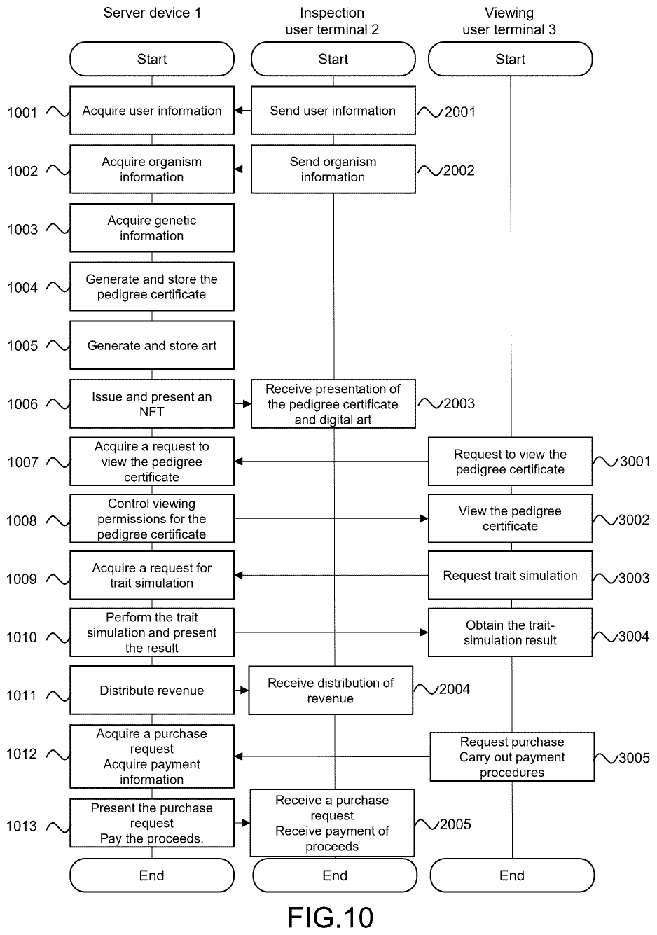

FIG. 10 is a diagram illustrating the operation of the transaction support system according to the present embodiment.

Server device 1 acquires user information from the inspection user terminal 2 (1001). Server device 1 acquires organism information from the inspection user terminal 2 (1002). Server device 1 acquires genetic information from the inspection device terminal 4 (1003). Server device 1 generates and stores a pedigree certificate (1004).

Server device 1 generates and stores digital art (1004). Server device 1 issues an NFT (1006). Server device 1 may present the pedigree certificate and the digital art to the inspection user terminal 2. Server device 1 acquires a request to view a pedigree certificate from the viewing user terminal 3 (1007). At this time, it may acquire a payment (viewing fee) from the viewing user who requests viewing. Server device 1 controls viewing permissions for the pedigree certificate and presents it to the viewing user (1008). Server device 1 acquires a request for trait simulation from the viewing user terminal 3 (1009). At this time, it may acquire a payment (simulation fee) from the viewing user who makes the request. Server device 1 performs a trait simulation and presents the result to the viewing user (1010). Server device 1 distributes revenue to the inspection user who holds the pedigree certificate that was the subject of the request to view, or that was the subject of the trait simulation (1011). Server device 1 acquires a purchase request for an organism from the viewing user terminal 3 and acquires payment information (1012). Server device 1 presents the purchase request for the organism and the payment information for the proceeds to the inspection user terminal 2 (1013).

As described above, according to the transaction support system of the present embodiment, it is possible to purchase an organism after viewing a pedigree certificate whose non-modification (no alteration) has been certified, and after viewing a prediction of traits in the case where the organism is crossed with another organism.

While preferred embodiments of the present disclosure have been described in detail with reference to the accompanying drawings, the technical scope of the present disclosure is not limited to such examples. It will be apparent to those having ordinary skill in the art that various modifications and alterations can be conceived within the spirit and scope of the technical idea set forth in the claims, and it is understood that such modifications and alterations also fall within the technical scope of the present disclosure.

The device described in this specification may be implemented as a standalone device, or may be implemented by multiple devices (e.g., cloud servers) connected via a network, in whole or in part. For example, the CPU and the storage device of server device 1 may be implemented by different servers that are connected to each other via a network.

The series of processes performed by the device described in this specification may be implemented using software, hardware, or any combination of software and hardware. A computer program for implementing the respective functions of server device 1 according to the present embodiment can be created and installed on a PC or the like. In addition, a computer-readable recording medium storing such a computer program can be provided. Examples of the recording medium include a magnetic disk, an optical disk, a magneto-optical disk, and flash memory. Further, the computer program may be distributed via a network, for example, without using a recording medium.

In addition, the processes described in this specification need not necessarily be executed in the order described. Some processing steps may be executed in parallel. Further, additional processing steps may be employed, and some processing steps may be omitted.

Furthermore, the effects described in this specification are merely descriptive or illustrative and are not limiting. That is, the technology according to the present disclosure can achieve other effects that would be apparent to those skilled in the art from the description of this specification, in addition to or in place of the above effects.

### EXPLANATION OF REFERENCE NUMERALS

1 Server device
2 Inspection user terminal
3 Viewing user terminal
NW Network
101 Processor
102 Memory
103 Storage device
104 Communication interface
105 Input device
106 Output device
111 User information acquisition unit
112 Organism information acquisition unit
113 Genetic information acquisition unit
114 Pedigree certificate generation unit
115 Digital art generation unit
116 NFT issuance unit
117 Presentation unit
118 Trait simulation unit
119 Distribution unit
120 Purchase/sale support unit
131 User information storage unit
132 Organism information storage unit
133 Genetic information storage unit
134 Pedigree certificate information storage unit
135 Digital art storage unit

1. A transaction support device for supporting transactions of organisms, the device comprising:
a genetic information storage unit configured to store genetic information of the organism; and
an NFT issuance unit configured to generate at least one of a pedigree certificate and digital art based on the genetic information, and to record, in a blockchain network, a user ID identifying a holder of the organism in association with a non-fungible token (NFT) associated with at least one of the pedigree certificate and the digital art,
wherein the NFT issuance unit is configured to generate the digital art based on information of a nucleotide sequence included in the genetic information.

2. (canceled)

3. The transaction support device according to claim 1, comprising a trait simulation unit configured to predict a trait of offspring when a first organism and a second organism are crossed, based on trait information of the first organism and the second organism and on the genetic information.

4. The transaction support device according to claim 3, comprising a distribution unit configured to, when revenue is obtained by allowing a viewing user who wishes to view the pedigree certificate to view the pedigree certificate, distribute a portion of the revenue to an inspection user who is the owner of the pedigree certificate.

5. A transaction support program for supporting transactions of organisms, the program causing a processor to execute:
a genetic information storing step of storing genetic information of the organism; and
an NFT issuing step of generating at least one of a pedigree certificate and digital art based on the genetic information, and recording, in a blockchain network, a user ID identifying a holder of the organism in association with an NFT associated with at least one of the pedigree certificate and the digital art,
wherein the NFT issuing step generates the digital art based on information of a nucleotide sequence included in the genetic information.

6. A transaction support method for supporting transactions of organisms, the method comprising, by a processor:
storing genetic information of the organism; and
generating at least one of a pedigree certificate and digital art based on the genetic information, and recording, in a blockchain network, a user ID identifying a holder of the organism in association with an NFT associated with at least one of the pedigree certificate and the digital art;
wherein the NFT issuing step generates the digital art based on information of a nucleotide sequence included in the genetic information.
